(19) United States
(12) Patent Application Publication
(10) Pub. No.: US 2004/0059409 A1
(43) Pub. Date: Mar. 25, 2004

(54) Method of applying coatings to a medical device

(76) Inventor: Eric B. Stenzel, Tuan (IE)

Correspondence Address: JONES DAY, 222 EAST 41ST STREET, NEW YORK, NY, 10017, US

(21) Appl. No.: 10/253,603

(22) Filed: Sep. 24, 2002

(51) Int. Cl. A61F 2/06

(52) U.S. Cl. 623/1.15

(57) ABSTRACT

A medical device, such as a stent, for delivering a biologically active material to body tissue of a patient and a method of making such a medical device are disclosed. The medical device has a tubular sidewall, wherein the sidewall is comprised of a plurality of struts each having an outer surface, an inner surface opposite the outer surface, and at least one side surface adjacent to and connecting the outer and inner surfaces. The outer surface or the inner surface is covered at least in part with a first coating comprising a first polymeric material and a first coating material, such as a biologically active material, e.g., paclitaxel, paclitaxel analogues, paclitaxel derivatives, or a combination thereof. The outer surface or inner surface not covered with the first coating is covered with a second coating comprising a second polymeric material and that is substantially free of the first coating material. Preferably, the first coating is applied to the outer surface of the strut. The disclosed method allows for greater efficiency and control in applying a biologically active material on the medical device and reduces patient exposure to unnecessary amounts of the biologically active material.

### FIELD OF THE INVENTION

The invention relates generally to a medical device that is useful for delivering a biologically active material to the body tissue of a patient, and the method for making such a medical device. More particularly, the invention is directed to a medical device having a tubular sidewall in which the inner and outer surfaces of the tubular sidewall are coated with two different coatings. One coating comprises a first coating material such as a biologically active material dispersed in a first polymeric material, while the other coating is substantially free of the first coating material.

### BACKGROUND OF THE INVENTION

Medical devices, such as implanted stents, have been coated with compositions comprising a biologically active material. One method of applying coatings loaded with a biologically active material to stents and other medical devices having a tubular portion is to coat the inside, sides, and outside of the tubular portion of the medical device with the composition to form a continuous coating on the tubular portion. See, e.g., U.S. Pat. No. 6,099,562 to Ding et al; U.S. Pat. No. 5,879,697 to Ding et al.; and U.S. Pat. No. 5,304,121 to Sahatjian. A reason for coating all these surfaces of the tubular portion of the medical device with a coating is to ensure adherence of the applied coating to the tubular portion. For example, when the tubular portion is comprised of struts, by applying coating to all surfaces of the struts of the tubular portion will form a coating that wraps around the struts. The fact that the coating “wraps around” the struts enhances adherence of the coating to the tubular portion.

However, in many medical devices all of the surfaces of the medical device or portions thereof do not need to be coated with a coating comprising a biologically active material. For instance, in medical devices having a tubular portion, such as a vascular stent, the inner surface and side surfaces of the tubular portion do not have to be coated with a coating containing a biologically active material. This is because these parts of the stent do not come in direct contact with the body lumen wall and do not apply the biologically active material to the body lumen wall. Therefore, it is not necessary to coat the inner surface and sides of the stent with a coating containing a biologically active material that is being applied to the body lumen wall.

Morever, in order to deliver certain medical devices, such as a balloon expandable stent, comprising a sidewall having struts, the stent must be put in its unexpanded state or “crimped” before it is delivered to a body lumen. Crimping can cause the coating to be torn or ripped off the struts. Specifically, if the first coating that is applied to the side surfaces of the struts of the stent contains polymeric materials that are relatively soft or sticky, then the coating will have a tendency to adhere to the side surfaces of adjacent struts during the crimping process. Such adherence will cause the coating to be ripped off the surfaces. Also, if the coating that is applied to the inner surface of the struts, which contacts the balloon, is coated with a material that is relatively soft or sticky, such coating will tend to be ripped off the inner surface because the coating will stick to the balloon as it contacts the inner surface during expansion. Therefore, there are problems associated with using relatively soft coating polymeric material. However, to form a coating containing a biologically active material, it is desirable to use such relatively soft polymeric material because such materials have a better ability to incorporate the biologically active material.

Accordingly, there is a need for more efficient methods of coating a medical device having a sidewall comprised of struts, that can more accurately deliver the desired dosage of a biologically active material from the coating of the device in order to limit patient exposure to excess drug in the coating. Furthermore, there is a need for a coated expandable stent comprising struts in which the undesired removal of coating from the stent, especially from the side surfaces of the struts during crimping of the stent and from the inner surface during expansion of the balloon, is minimized. There is also a desire for a stent comprised of struts and having a continuous coating that remains adhered to the struts during crimping and expansion, but which contains a minimal amount of biologically active material contained therein, and a method of coating such a stent. There is also a need for a method of coating a pre-fabricated stent with two different coatings to form a continuous coating disposed on the struts of the stent.

### SUMMARY OF THE INVENTION

These and other objectives are accomplished by the present invention. The present invention provides a coated medical device. The medical device comprises a tubular sidewall comprising a plurality of struts each having an outer surface, an inner surface opposite the outer surface, and at least one side surface adjacent to the outer and inner surfaces and connecting the outer surface and inner surface. The outer surface or the inner surface is covered at least in part by a first coating which comprises a first coating material dispersed in a first polymeric material. The outer surface or the inner surface, that is not covered by the first coating, and the side surface are covered at least in part by a second coating comprising a second polymeric material that is substantially free of the first coating material. The first coating and the second coating form a continuous coating disposed on the struts. In other words, the first and second coatings are connected to each other in at least one location. In addition, the surface covered with the first coating is substantially free of the second coating, and the surface covered with the second coating is substantially free of the first coating.

In an alternative embodiment, an expandable stent comprises a tubular sidewall comprising a plurality of struts each having an outer surface, an inner surface opposite the outer surface, and at least one side surface adjacent to the outer and inner surfaces, and connecting the outer surface and inner surface. The outer surface is covered at least in part by a first coating which comprises a biologically active material dispersed in a first polymeric material. The biologically active material can be selected from the group consisting of paclitaxel, paclitaxel analogues, paclitaxel derivatives, and combinations thereof. The inner surface and the side surface are covered at least in part by a second coating comprising a second polymeric material. The inner surface and side surface are substantially free of the biologically active material. The first coating and the second coating form a continuous coating disposed on the struts.

In another embodiment, a method of coating a medical device is disclosed. Specifically, the method of the present invention comprises providing a pre-fabricated medical device having a tubular sidewall, wherein the sidewall comprises a plurality of struts each having an outer surface, an inner surface opposite the outer surface, and at least one side surface adjacent to the outer and inner surface and connecting the outer surface and inner surface. The method comprises applying to the outer surface or the inner surface a first coating comprising a first coating material dispersed in a first polymeric material; and applying to the outer surface or the inner surface, that is not covered by the first coating, and the side surface a second coating comprising a second polymeric material, wherein the second coating is substantially free of the first coating material. The first coating and the second coating form a continuous coating disposed on the struts. In addition, the surface covered with the first coating is substantially free of the second coating, and the surface covered with the second coating is substantially free of the first coating. This method may further comprise masking either the inner or outer surface during application of the coating to the opposing surface.

In yet another embodiment, a method of coating an expandable stent having a sidewall, wherein the sidewall comprises a plurality of struts each having an outer surface, an inner surface opposite the outer surface, and at least one side surface adjacent to the outer and inner surfaces and connecting the outer surface and the inner surface. The method comprises applying to the outer surface a first coating comprising a biologically active material dispersed in a first polymeric material. The method further comprises applying to the inner surface and the side surface a second coating comprising a second polymeric material, wherein the second coating is substantially free of the biologically active material. The first coating and the second coating form a continuous coating disposed on the struts. In addition, the surface covered with the first coating is substantially free of the second coating, and the surface covered with the second coating is substantially free of the first coating. In this embodiment, the biologically active material is selected from the group consisting of paclitaxel, paclitaxel analogues, paclitaxel derivatives, and combinations thereof.

The methods of the present invention provide a more efficient process for applying a coating comprising a first coating material such as a biologically active material dispersed in a polymeric material to the surface of a medical device. Specifically, the devices and methods of the present invention provide a more efficient means of administering a biologically active material to a patient. In one embodiment, because only the surface of the device or struts, i.e., the outer surface, that is in contact with the body tissue that is to be treated with the biologically active material is coated with a coating containing the biologically active material, there is no unnecessary biologically active material included on the surfaces of the device or struts that are not in contact with the body tissue to be treated. Hence, the patient is not exposed to an amount of biologically active material in excess of the desired or prescribed dosage. Also, because an unnecessary and excess amount of biologically active material is not included in the coating of the medical device, the cost of manufacturing the device can be reduced.

Furthermore, in the present invention, the polymeric material in the coating applied to the inner and side surfaces of the struts of the stent can differ from the polymeric material in the coating applied to the outer surface of the struts of the stent. Therefore, the polymeric material in the coating applied to the inner and side surfaces can be selected from relatively “harder” or “less sticky” polymeric materials. Such polymeric materials are likely to reduce the undesired tearing or ripping of the coating from the side surfaces during crimping of the stent when the coating on the side surfaces of adjacent struts can contact each other. Also, the application of harder polymeric materials to the inner surface of the struts of a balloon expandable stent or tubular portion of the medical device can reduce the chances that the coating will stick to the balloon when it expands and contacts the inner surface.

### BRIEF DESCRIPTION OF THE DRAWINGS

FIG. 1 depicts a perspective view of an implantable stent, having a sidewall comprising a plurality of struts with an outer surface, an inner surface, and side surfaces, that is useful in an embodiment of the present invention.

FIG. 1A shows the outer surface, inner surface, and side surface of a strut of the implantable stent of FIG. 1.

FIG. 1B shows a cross-section of the stent of FIG. 1.

FIG. 2 depicts a longitudinal cross-section of a coated stent strut that is useful in an embodiment of the present invention.

FIG. 3 depicts an individual rounded strut of a medical device that is useful in an embodiment of the present invention. The strut is coated with a first coating and a second coating.

FIG. 3A shows a cross-sectional view of the individual rounded strut depicted in FIG. 3.

### DESCRIPTION OF THE PREFERRED EMBODIMENTS

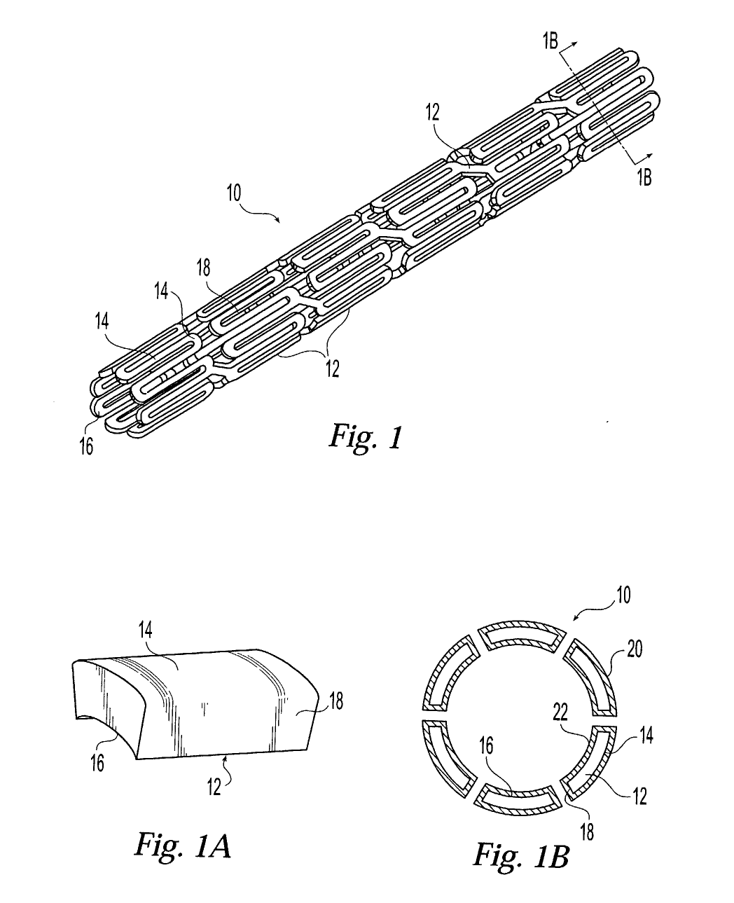

The medical devices suitable for the present invention comprise a tubular sidewall 10. Such a sidewall 10 is preferably comprised of a plurality of struts 12. The struts 12 may be arranged in any suitable configuration. Also, the struts 12 do not all have to have the same shape or geometric configuration. Each individual strut 12 has an outer surface 14 adapted for exposure to the body tissue of the patient, an inner surface 16 opposite the outer surface 14, and at least one side surface 18 adjacent to the outer surface 14 and inner surface 16. The tubular sidewall 10 may be a stent, as shown in FIG. 1 which shows the inner surface 16, outer surface 14, and side surface 18 of the individual struts 12.

A strut 12 can generally be considered to be comprised of four surfaces: an outer surface 14, an inner surface 16, and two side surfaces 18 connecting the outer surface 14 and the inner surface 16. FIG. 1A is an enlarged view of a section of a strut 12 depicted in FIG. 1. This figure shows the outer surface 14, the inner surface 16, and one side surface 18 of the strut 12. The outer surface 14 of the strut 12 is the surface that comes in direct contact with the body lumen wall when the medical device is implanted. The outer surface 14 need not include only one flat surface or facet. Instead, it can be rounded, such as in the case of a wire strut 12, or have a number of facets. The inner surface 16 of the strut 12 is the surface that is opposite the outer surface 14. The two side surfaces 18 are the surfaces of the strut 12 that are adjacent to the inner surface 16 or outer surface 14. The side surface 18 connects the inner surface 16 and the outer surface 14. Like the outer surface 14, the inner surface 16 and side surface 18 can be rounded or have a number of facets. FIG. 1B is a cross-section of the stent of FIG. 1 and shows the inner surface 16, the two side surfaces 18, and the outer surface 14 of the struts 12 of the stent.

Suitable medical devices that can have tubular sidewalls 10 made of struts 12 include, but are not limited to, stents, surgical staples, and vascular or other grafts. Other suitable medical devices may also include devices that do not necessarily have tubular sidewalls 10 made of struts 12, but have an inner surface 16 and an outer surface 14. Such medical devices include, but are not limited to, catheters, such as central venous catheters and arterial catheters, guidewires, cannulas, cardiac pacemaker leads or lead tips, cardiac defibrillator leads or lead tips, implantable vascular access ports, blood storage bags, blood tubing, vascular or other grafts, intra-aortic balloon pumps, heart valves, cardiovascular sutures, total artificial hearts and ventricular assist pumps, extra-corporeal devices such as blood oxygenators, blood filters, hemodialysis units, hemoperfusion units or plasmapheresis units.

Medical devices which are particularly suitable for the present invention include any stent for medical purposes, which are known to the skilled artisan. Suitable stents include, for example, vascular stents such as self-expanding stents and balloon expandable stents. Examples of self-expanding stents are illustrated in U.S. Pat. Nos. 4,655,771 and 4,954,126 issued to Wallsten and U.S. Pat. No. 5,061,275 issued to Wallsten et al. Examples of appropriate balloon-expandable stents are shown in U.S. Pat. No. 5,449,373 issued to Pinchasik et al.

The framework of the suitable stents may be formed through various methods as known in the art. The framework may be welded, molded, laser cut, electro-formed, or consist of filaments or fibers which are wound or braided together in order to form a continuous structure.

The medical devices suitable for the present invention may be fabricated from polymeric and/or metallic materials. Suitable polymeric materials include without limitation polyurethane and its copolymers, silicone and its copolymers, ethylene vinyl-acetate, polyethylene terephtalate, thermoplastic elastomers, polyvinyl chloride, polyolefins, cellulosics, polyamides, polyesters, polysulfones, polytetrafluorethylenes, polycarbonates, acrylonitrile butadiene styrene copolymers, acrylics, polylactic acid, polyglycolic acid, polycaprolactone, polylactic acid-polyethylene oxide copolymers, cellulose, collagens, and chitins. Suitable metallic materials include metals and alloys based on titanium (such as nitinol, nickel titanium alloys, thermo-memory alloy materials), stainless steel, tantalum, nickel-chrome, or certain cobalt alloys including cobalt-chromium-nickel alloys such as Elgiloy® and Phynox®. Metallic materials also include clad composite filaments, such as those disclosed in WO 94/16646.

Preferably, the medical device is pre-fabricated before application of the coatings. The pre-fabricated medical device is in its final shape. For example, if the finished medical device is a stent having an opening in its sidewall 10, then the opening is formed in the device before application of the coatings.

At least a portion of the outer surface 14 of the medical device is coated with a coating that is different from the coating applied to at least a portion of the inner surface 16 and the side surface 18 of the stent or tubular sidewall 10 of the medical device. The outer surface 14 or inner surface 16 of the struts 12 forming the sidewall 10 of the stent or tubular portion is coated with a first coating 20 comprising a first coating material dispersed in a first polymeric material. The first coating material and the polymeric material are mixed together and preferably dissolved and/or suspended in an appropriate solvent before application to the surface.

The outer surface 14 or the inner surface 16 of the struts 12, that is not covered by the first coating 20, and the side surfaces 18 of the struts 12 is coated with a second coating 22 comprising a second polymeric material. The second polymeric material is also preferably dissolved or suspended in a solvent before application to a surface of the struts 12. The second coating 22 is substantially free of the first coating material present in the first coating 20. The entirety of the outer surface 14 or the inner surface 16 of the stent or tubular portion of the medical device can be coated or only discrete sections thereof. However, the first coating 20 and second coating 22 should be connected together in at least one location to form a continuous coating disposed on the struts 12. In other words, the struts 12 forming the sidewall 10 of the stent or tubular portion should be covered by a continuous coating in which the coating covering the inner surface 16 is connected to the coating covering the outer surface 14 by the coating that covers at least one side surface 18 of the struts 12. Such a continuous coating wraps around the struts 12 and improves the adherence of the two coatings to the struts 12 and hence the stent. The surface covered with the first coating 20 is substantially free of the second coating 22, and the surface covered with the second coating 22 is substantially free of the first coating 20.

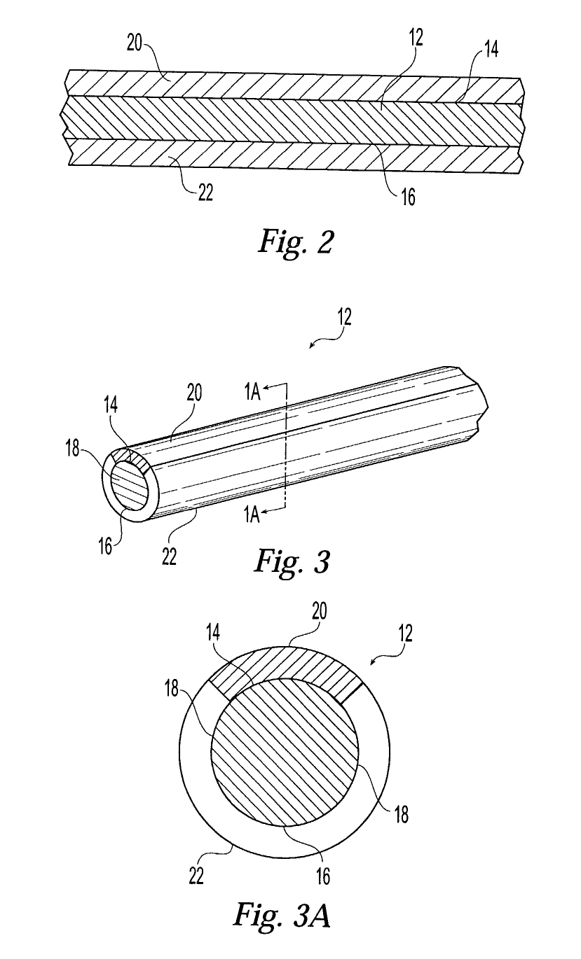

FIG. 2 shows a longitudinal cross-section of a strut 12 of an expandable stent having a sidewall 10 with an outer surface 14 that is adapted for exposure to a body lumen and an inner surface 16 opposite the outer surface 14. In this embodiment, the strut 12 has been coated with two different coatings. The outer surface 14 is coated with a first coating 20 and the inner surface 16 is coated with a second coating 22. FIG. 1B shows the cross-sections of the stent of FIG. 1 in which the individual struts 12 comprising the sidewall 10 have been coated with the coatings. The outer surface 14 is coated with the first coating 20, and the inner surface 16 and side surfaces 18 are coated with the second coating 22. The coatings are connected and adjacent to each other to form a continuous coating disposed on the strut 12.

FIG. 3 shows an individual strut 12 that is rounded. The surface that contacts the body lumen is the outer surface 14. Opposite the outer surface 14 is the inner surface 16 of the strut 12. The two surfaces that are generally between the outer surface 14 and inner surface 16 are the side surfaces 18. One side surface is shown in FIG. 3. The strut 12 has a first coating 20 on the outer surface 14 and a second coating 22 on the inner surface 16 and side surfaces 18. FIG. 3A shows a cross-sectional view of the strut 12. In FIG. 3A, the first coating 20 covers the outer surface 14 and the second coating 22 covers the inner surface 16 and two side surfaces 18. At the point where the outer surface 14 is adjacent to and contacts the side surfaces 18, the first coating 20 and the second coating 22 connect together to form a continuous coating disposed on the strut 12.

Preferably, the first coating material is a biologically active material. The term “biologically active material” encompasses therapeutic agents, such as drugs, and also genetic materials and biological materials. Suitable genetic materials include DNA or RNA, such as, without limitation, DNA/RNA encoding a useful protein and DNA/RNA intended to be inserted into a human body including viral vectors and non-viral vectors. Suitable viral vectors include adenoviruses, gutted adenoviruses, adeno-associated virus, retroviruses, alpha virus (Semliki Forest, Sindbis, etc.), lentiviruses, herpes simplex virus, ex vivo modified cells (e.g., stem cells, fibroblasts, myoblasts, satellite cells, pericytes, cardiomyocytes, sketetal myocytes, macrophage), replication competent viruses (e.g., ONYX-015), and hybrid vectors. Suitable non-viral vectors include artificial chromosomes and mini-chromosomes, plasmid DNA vectors (e.g., pCOR), cationic polymers (e.g., polyethyleneimine, polyethyleneimine (PEI)) graft copolymers (e.g., polyether-PEI and polyethylene oxide-PEI), neutral polymers PVP, SP1017 (SUPRATEK), lipids or lipoplexes, nanoparticles and microparticles with and without targeting sequences such as the protein transduction domain (PTD).

Suitable biological materials include cells, yeasts, bacteria, proteins, peptides, cytokines and hormones. Examples of suitable peptides and proteins include growth factors (e.g., FGF, FGF-1, FGF-2, VEGF, Endotherial Mitogenic Growth Factors, and epidermal growth factors, transforming growth factor α and β, platelet derived endothelial growth factor, platelet derived growth factor, tumor necrosis factor α, hepatocyte growth factor and insulin like growth factor), transcription factors, proteinkinases, CD inhibitors, thymidine kinase, and bone morphogenic proteins (BMP's), such as BMP-2, BMP-3, BMP-4, BMP-5, BMP-6 (Vgr-1), BMP-7 (OP-1), BMP-8. BMP-9, BMP-10, BMP-11, BMP-12, BMP-13, BMP-14, BMP-15, and BMP-16. Currently preferred BMP's are BMP-2, BMP-3, BMP-4, BMP-5, BMP-6, BMP-7. These dimeric proteins can be provided as homodimers, heterodimers, or combinations thereof, alone or together with other molecules. Cells can be of human origin (autologous or allogeneic) or from an animal source (xenogeneic), genetically engineered, if desired, to deliver proteins of interest at the transplant site. The delivery media can be formulated as needed to maintain cell function and viability. Cells include whole bone marrow, bone marrow derived mono-nuclear cells, progenitor cells (e.g., endothelial progentitor cells) stem cells (e.g., mesenchymal, hematopoietic, neuronal), pluripotent stem cells, fibroblasts, macrophage, and satellite cells.

Biologically active material also includes non-genetic therapeutic agents, such as: anti-thrombogenic agents such as heparin, heparin derivatives, urokinase, and PPack (dextrophenylalanine proline arginine chloromethylketone); anti-proliferative agents such as enoxaprin, angiopeptin, or monoclonal antibodies capable of blocking smooth muscle cell proliferation, hirudin, and acetylsalicylic acid, amlodipine and doxazosin; anti-inflammatory agents such as glucocorticoids, betamethasone, dexamethasone, prednisolone, corticosterone, budesonide, estrogen, sulfasalazine, and mesalamine; antineoplastic/antiproliferative/anti-miotic agents such as paclitaxel, 5-fluorouracil, cisplatin, vinblastine, vincristine, epothilones, methotrexate, azathioprine, adriamycin and mutamycin; endostatin, angiostatin and thymidine kinase inhibitors, taxol and its analogs or derivatives; anesthetic agents such as lidocaine, bupivacaine, and ropivacaine; anti-coagulants such as D-Phe-Pro-Arg chloromethyl keton, an RGD peptide-containing compound, heparin, antithrombin compounds, platelet receptor antagonists, anti-thrombin antibodies, anti-platelet receptor antibodies, aspirin (aspirin is also classified as an analgesic, antipyretic and anti-inflammatory drug), dipyridamole, protamine, hirudin, prostaglandin inhibitors, platelet inhibitors and tick antiplatelet peptides; vascular cell growth promotors such as growth factors, Vascular Endothelial Growth Factors (FEGF, all types including VEGF-2), growth factor receptors, transcriptional activators, and translational promoters; vascular cell growth inhibitors such as antiproliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents, vasodilating agents, and agents which interfere with endogenous vasoactive mechanisms; anti-oxidants, such as probucol; antibiotic agents, such as penicillin, cefoxitin, oxacillin, tobranycin; angiogenic substances, such as acidic and basic fibrobrast growth factors, estrogen including estradiol (E2), estriol (E3) and 17-Beta Estradiol; and drugs for heart failure, such as digoxin, beta-blockers, angiotensin-converting enzyme (ACE) inhibitors including captopril and enalopril.

Preferred biologically active materials include anti-proliferative drugs such as steroids, vitamins, and restenosis-inhibiting agents. Preferred restenosis-inhibiting agents include microtubule stabilizing agents such as Taxol, paclitaxel, paclitaxel analogues, derivatives, and mixtures thereof. For example, derivatives suitable for use in the present invention include 2′-succinyl-taxol, 2′-succinyl-taxol triethanolamine, 2′-glutaryl-taxol, 2′-glutaryl-taxol triethanolamine salt, 2′-O-ester with N-(dimethylamino ethyl) glutamine, and 2′-O-ester with N-(dimethylaminoethyl) glutamide hydrochloride salt.

Other preferred biologically active materials include nitroglycerin, nitrous oxides, antiobitics, aspirins, digitalis, and glycosides.

The amount of the first coating material such as a biologically active material present in the first coating 20 can be adjusted to meet the needs of the patient. In general, the amount of the biologically active material used may vary depending on the application or biologically active material selected. In addition, the quantity of biologically active material used may be related to the selection of the polymer carrier. One of skill in the art would understand how to adjust the amount of a particular biologically active material to achieve the desired dosage or amount.

Preferably, the first coating 20 is applied to the outer surface 14 and the biologically active material contained in the first coating 20 is preferably useful in treating the body tissue that comes in contact with the outer surface 14, e.g., such as an antiproliferative drug.

Since the inner surface 16 of the medical device sidewall 10 does not come in direct contact with the body tissue to be treated, such as a body lumen wall, there is generally no need for this biologically active material to be present in the coating on the inner surface 16. Thus, in such a medical device the biologically active material that is to be applied to the body tissue is applied to the outer surface 14 of the stent, which is the surface that is exposed to the body tissue.

Applying a coating comprising a biologically active material for treating the body lumen wall tissue to primarily the surface of the struts 12 that directly contacts the body lumen wall tissue to be treated results is a cost savings because unnecessary amounts of such biologically active materials are not applied to the inner surface 16 that does not directly contact the body lumen wall tissue. Also, the patient is not exposed to unnecessary dosages of such biologically active materials.

Moreover, in one embodiment not only is the coating on the inner surface 16 substantially free of the biologically active material present in the coating on the outer surface 14, but also the coating on the inner surface 16 is substantially free of any biologically active material.

In another embodiment, although the coating on the inner surface 16 of the struts 12 is substantially free of the biologically active material present in the coating of the outer surface 14 of the struts 12, the coating on the inner surface 16 may comprise a different biologically active material than the one in the coating on the outer surface 14. For example, in one embodiment, the second coating 22 covering the inner surface 16 may include an anti-thrombogenic agent such as clotting inhibitor or a blood-thinning agent to prevent thrombosis such as Heparin or Abciximab (Reopro). In such case, the stent or other medical device may serve as a treatment for restenosis coupled with protection from the creation of blood clots and thrombosis being formed from the edges of the stent.

Both the first coating 20 and the second coating 22 comprise at least one polymeric material. Although the invention can be practiced by using a single type of polymeric material in each coating, various combinations of polymers can be employed to form each coating. The appropriate mixture of polymers can be coordinated with the first coating materials of interest to produce desired effects when coated on a medical device. The first coating material such as a biologically active material is dispersed in the polymeric materials.

Moreover, the polymeric material of both coatings should be a material that is biocompatible and avoids irritation to body tissue and damage to the lumen wall. Preferably, the polymeric materials used in the first coating 20 and the second coating 22 are selected from the following: polyurethanes, silicones (e.g., polysiloxanes and substituted polysiloxanes), and polyesters. Also preferable as a polymeric material is styrene-isobutylene-styrene (SIBS). Other polymers which can be used include ones that can be dissolved and cured or polymerized on the medical device or polymers having relatively low melting points that can be blended with biologically active materials. Additional suitable polymers include, thermoplastic elastomers in general, polyolefins, polyisobutylene, ethylene-alphaolefin copolymers, acrylic polymers and copolymers, vinyl halide polymers and copolymers such as polyvinyl chloride, polyvinyl ethers such as polyvinyl methyl ether, polyvinylidene halides such as polyvinylidene fluoride and polyvinylidene chloride, polyacrylonitrile, polyvinyl ketones, polyvinyl aromatics such as polystyrene, polyvinyl esters such as polyvinyl acetate, copolymers of vinyl monomers, copolymers of vinyl monomers and olefins such as ethylene-methyl methacrylate copolymers, acrylonitrile-styrene copolymers, ABS (acrylonitrile-butadiene-styrene) resins, ethylene-vinyl acetate copolymers, polyamides such as Nylon 66 and polycaprolactone, alkyd resins, polycarbonates, polyoxymethylenes, polyimides, polyethers, epoxy resins, rayon-triacetate, cellulose, cellulose acetate, cellulose butyrate, cellulose acetate butyrate, cellophane, cellulose nitrate, cellulose propionate, cellulose ethers, carboxymethyl cellulose, collagens, chitins, polylactic acid, polyglycolic acid, polylactic acid-polyethylene oxide copolymers, EPDM (ethylene-propylene-diene) rubbers, fluorosilicones, polyethylene glycol, polysaccharides, phospholipids, and combinations of the foregoing.

More preferably for medical devices which undergo mechanical challenges, e.g. expansion and contraction, the polymeric materials should be selected from elastomeric polymers such as silicones (e.g. polysiloxanes and substituted polysiloxanes), polyurethanes, thermoplastic elastomers, ethylene vinyl acetate copolymers, polyolefin elastomers, and EPDM rubbers. Because of the elastic nature of these polymers, the coating adheres better to the surface of the medical device when the device is subjected to forces, stress or mechanical challenge.

The amount of the polymeric material present in the coatings can vary based on the application for the medical device. One skilled in the art is aware of how to determine the desired amount and type of polymeric material used in the coatings.

Preferably, when the struts 12 of the stent are made of a biodegradable material, the polymeric materials used in the coatings covering the surfaces of the struts 12 should also be biodegradable so that the coating degrades with the strut 12 material. However, when the strut 12 is a form of a non-biodegradable material such as a metallic material the polymeric material used in the coating for covering the outer surface 14 of the struts 12 should be biostable.

Furthermore, the polymeric material used in the coating covering the inner surface 16 and side surfaces 18 of the struts 12 is preferably “harder” or “less sticky” as compared to the polymeric material used in the coating covering the outer surface 14. The terms “harder” and “less sticky” mean that the polymeric material has a greater resistance to mechanical damage or is less malleable and has a reduced ability to bond to other surfaces when contacted with such other surfaces. Also, when the polymeric material of the coating covering the inner surface 16 is “harder” or “less sticky” such coating will be less likely to adhere to the balloon when the balloon contacts the inner surface 16 during expansion. A polymeric material that is less malleable is less likely to deform when a force is applied to it. Therefore, such a polymeric material has a lesser tendency to adhere to other polymeric materials. Therefore, when a “harder” or “less sticky” polymeric material is used in a coating covering the side surfaces 18 of the struts 12 and the struts 12 are crimped, there is a greater likelihood that even if the struts 12 contact each other their coatings will not stick to each other and rip or tear when the struts 12 are expanded.

Moreover, the polymeric material in the second coating 22 may be flexible enough to stretch during deployment, and be more apt to resist the stresses of the crimping process than that of the first coating 20. In addition, the second polymeric material preferably has a higher shear strength than the first polymeric material in order to provide enhanced adhesion to the stent.

Before applying the coatings to the medical device, the constituents of the coating, e.g., polymeric material, first coating material, should be dissolved or suspended in a solvent to form a coating composition. The solvent used with the first coating 20 may be the same or different than the solvent used with the second coating 22. One or more solvents may be used with each coating. Suitable solvents are ones which can dissolve the polymeric material into solution or form dispersions of the polymeric material in the solvent. If a biologically active material is present, the solvent preferably can also dissolve or suspend the biologically active material. Any solvent which does not alter or adversely impact the therapeutic properties of the biologically active material can be employed in the method of the present invention. Examples of suitable solvents include, but are not limited to, tetrahydrofuran, methylethylketone, chloroform, toluene, acetone, isooctane, 1,1,1,-trichloroethane, and mixture thereof.

The polymeric material and biologically active material are mixed together with the solvents and then applied to the stent. After the coating has been applied, the solvents are evaporated from the coating leaving the mixture of the polymeric material and the biologically active material on the struts 12. The biologically active material is dispersed in the polymeric material. The polymer material is porous in form and allows the dispersed biologically active agent to pass through the pores in order to be released to the desired body tissue. Factors influencing the release of the biologically active material include, but are not limited to, the polymer or carrier selection, the solvent used, and the biologically active agent selected. Also, the use of a polymeric material allows for a time release profile to be created to release the dose over a desired period of time. One of ordinary skill in the art would understand how to adjust these factors to obtain a desirable release profile.

The present invention also comprises a method of making a medical device coated with two different coatings. Generally, the method of making the medical device of the present invention comprises providing a medical device comprising a tubular portion having a sidewall 10. The sidewall 10 comprises a plurality of struts 12 each having an outer surface 14, an inner surface 16 opposite the outer surface 14, and at least one side surface adjacent to and connecting the inner surface 16 and the outer surface 14. The method comprises applying to the outer surface 14 or the inner surface 16 a first coating 20 which includes a first coating material dispersed in a polymer material. The method further comprises applying a second coating 22 to either the outer surface 14 or inner surface 16 that is not covered by the first coating 20 and to the side surfaces 18. The first coating 20 and the second coating 22 are connected to each other in at least one location to form a continuous coating disposed on the struts 12.

The medical device is coated by any suitable method as known by one skilled in the art. The outer surface 14 and the inner and side surfaces 18 may be coated by the same or different methods. Suitable methods of coating the medical device include, but are not limited to, spray-coating, painting, rolling, electrostatic deposition, or a combination thereof. In a preferred embodiment, the coatings are applied consecutively. The coatings may be applied in any order. The coating may be applied one or more times to a surface. Preferably, the outer surface 14 is first coated by rolling or transfer coating, and the inner surface 16 is coated by spray-coating. Also, an expandable stent is preferably coated in the expanded position.

In one embodiment, the first coating 20 can be deposited onto a substrate. Then, the outer surface of the tubular portion of the medical device may be rolled over the coated substrate to transfer the coating to the outer surface 14 of the struts making up the tubular portion. In addition, the sidewall of a pre-fabricated stent may be placed over a rigid mandrel to protect the stent from mechanical damage and hold the stent in place. The stent which is mounted on the mandrel may then be rolled over the coated baseplate or substrate to transfer the first coating 20 to the outer surface of the struts 12 of the stent 10.

For example, the first coating 20 composition may be deposited on a substrate, and a stent 10 may be rolled over the coated substrate to transfer the first coating 20 composition to the outer surface 14 of the struts 12 of the stent 10. The substrate is preferably made from materials that provide minimal adhesion force to the first coating 20 composition so that the first coating 20 composition can be easily removed and transferred to the stent 10.

This first layer of the first coating 20 can be cured or dried and the process repeated until the required thickness of the first coating 20 is achieved. The first coating 20 may be cured using infrared light. The first coating 20 may optionally be re-wetted and dried with a solvent in order to provide a more uniform surface texture.

Preferably, the inner surface 16 and side surfaces 18 of stent 10 are coated by a spraying process. For example, a nozzle assembly may be used to spray the second coating 22 composition onto the inner surface 16 of the sidewall 10 of a stent 10. The nozzle assembly may be in the form of a cone that sprays the coating composition at an angle. The angle of the spray from the nozzles may need to be adjusted to ensure uniform thickness of the coating on the inner surface 16. Also, a nozzle assembly with small spray nozzles can be inserted into one end of the stent 10 and moved through the stent 10 until it extends past the opposite end of the stent 10. Preferably, the spray mist flow is started while the nozzle is still outside of the stent 10. This step places a coating composition on the inside surface and one side surface of the struts 12 of the stent. Optionally, the second coating 22 is cured or dried. The coating process may be repeated again. Preferably, the spray nozzle is inserted into the other end of the stent to coat the other side surface of the struts 12 as well as providing an additional layer on the inner surface 16 of the strut 12. By repeating the spraying from two directions, both side surfaces 18 are coated with a coating that connects with the coating on the outer surface 14. Thus, a continuous coating is formed.

Preferably, the second coating 22 is sprayed two times on the inner surfaces 16 and side surfaces 18 of the struts 12. The stent may then be fully cured or dried.

To ensure that the surface of a coating is sufficiently smooth, a coating can be applied on the surface of the sidewall 10 of the medical device. To smooth the coating, the coating can be re-wetted by applying a solvent. For example, the surface may be sprayed again with a solvent which is the same as or different from the solvent used to prepare the coating. The solvent will dissolve the coating applied to the surfaces and smooth out the surfaces of the coating. A rough surface can also be exposed to infrared heat to re-melt the coating to smooth the surface of the coating taking into consideration the effect of heat on the biologically active material.

Before application of a coating composition on one surface, the other surface may be masked. A surface is masked, for instance, by application of a protective wrap to that surface. The protective wrap is a material that would protect the coated surface from exposure to the coating applied to the opposing surface. Suitable material for this protective wrap include, for example, PTFE film, dyna-leap, Kapton®, or any other appropriate type of covering or wrapping material. Thus, the outer surface 14 of the struts 12 may be masked during application of the second coating 22 composition to the inner surfaces 16 and side surfaces 18 of the struts 12. For example, during application of the second coating 22 composition to the inner surface 16 of the struts 12, the outer surface 14 of the struts 12 may be masked with a protective wrap. The protective wrap preferably extends for the length of the stent, and is secured so that it does not unwrap. The protective wrap serves to protect the outer surface 14 from exposure to the second coating 22 composition as it is being applied to the inner surface 16. Thus, the wrap will protect an outer surface 14 that has been already coated from additional deposition of the coating to be applied to the inner surfaces 16 and side surfaces 18. After the protective wrap has been applied to the outer surface 14 of the stent, a mandrel which may have been used during coating of the outer surface 14 is removed from the inside of the stent. After the inner surfaces 16 and side surfaces 18 of the struts 12 of the medical device have been coated, the wrap covering the outer surface 14 may be removed.

Also, the inner surfaces 16 and side surfaces 18 maybe masked during application of the first coating 20 to the outer surface 14 so that the inner surface 16 will be substantially free of the first coating material present in the coating on the outer surface 14.

After one or both of the coatings have been applied, the medical device may be cured or dried which will evaporate the solvent. Curing is defined as the process of converting the polymeric material into the finished or useful state by the application of heat, vacuum, and/or chemical agents which induce physico-chemical changes. The applicable time and temperature for curing are determined by the particular polymer involved and particular biologically active material used, if any, as known by one skilled in the art. The coated medical devices may thereafter be subjected to a post-cure process wherein the medical devices are exposed to a low energy for stabilization of the coating. Also, after the medical device is coated, it preferably should be sterilized by methods of sterilization as known in the art.

In use, a coated medical device, such as an expandable stent, according to the present invention can be made to provide desired release profile of the biologically active material. The medical devices and stents of the present invention may be used for any appropriate medical procedure. Delivery of the medical device can be accomplished using methods well known to those skilled in the art, such as mounting the stent on an inflatable balloon disposed at the distal end of a delivery catheter.

The description contained herein is for purposes of illustration and not for purposes of limitation. Changes and modifications may be made to the embodiments of the description and still be within the scope of the invention. Furthermore, obvious changes, modifications or variations will occur to those skilled in the art. Also, all references cited above are incorporated herein, in their entirety, for all purposes related to this disclosure.

I claim:

1. A coated medical device comprising:
a tubular sidewall, wherein the sidewall comprises a plurality of struts each having an outer surface, an inner surface opposite the outer surface, and at least one side surface adjacent to the outer surface and the inner surface and connecting the inner surface and outer surface;
wherein the outer surface or the inner surface has at least a portion that is covered by a first coating comprising a first coating material dispersed in a first polymeric material,
wherein both the side surface and the outer surface or the inner surface, that does not have at least a portion that is covered by the first coating, has at least a portion that is covered by a second coating comprising a second polymeric material and being substantially free of the first coating material,
wherein the first coating and the second coating form a continuous coating disposed on the struts, and
wherein the surface covered with the first coating is substantially free of the second coating and wherein the surface covered with the second coating is substantially free of the first coating.

2. The medical device of claim 1, wherein the tubular sidewall forms a tent.

3. The medical device of claim 1, wherein the first coating material is a biologically active material.

4. The medical device of claim 3, wherein the biologically active material is selected from the group consisting of anti-thrombogenic agents, anti-angiogenesis agents, anti-proliferative agents, growth factors, and radiochemicals.

5. The medical device of claim 4, wherein the anti-proliferative agent is selected from the group consisting of paclitaxel, paclitaxel analogues, paclitaxel derivatives, and combinations thereof.

6. The medical device of claim 1, wherein the first and second polymeric materials are selected from the group consisting of styrene-isobutylene-styrene, polyurethanes, silicones, polyesters, polyolefins, polyisobutylene, ethylene-alphaolefin copolymers, acrylic polymers and copolymers, vinyl halide polymers, polyvinyl ethers, polyvinylidene halides, polyacrylonitrile, polyvinyl ketones, polyvinyl aromatics, polyvinyl esters, copolymers of vinyl monomers, copolymers of vinyl monomers and olefins, polyamides, alkyd resins, polycarbonates, polyoxymethylenes, polyimides, polyethers, epoxy resins, polyurethanes, rayon-triacetate, cellulose, cellulose acetate, cellulose butyrate, cellulose acetate butyrate, cellophane, cellulose nitrate, cellulose propionate, cellulose ethers, carboxymethyl cellulose, collagens, chitins, polylactic acid, polyglycolic acid, polylactic acid-polyethylene oxide copolymers, ethylene-propylene-diene rubbers, fluorosilicones, polyethylene glycol, polysaccharides, phospholipids, and combinations thereof.

7. The medical device of claim 1, wherein the second polymeric material is the same as the first polymeric material.

8. The medical device of claim 1, wherein the inner surface is covered by the second coating and the second polymeric material is harder than the first polymeric material.

9. The medical device of claim 1, wherein the first and the second polymeric material are biocompatible.

10. The medical device of claim 1, wherein at least one of said coatings is biodegradable.

11. An expandable stent comprising:
a tubular sidewall, wherein the sidewall comprises a plurality of struts each having an outer surface that is adapted for exposure to a body lumen, an inner surface opposite the outer surface, and at least one side surface adjacent to the outer surface and the inner surface and connecting the inner surface and the outer surface; and
wherein at least a portion of the outer surface is covered by a first coating comprising a biologically active material dispersed in a first polymeric material, wherein the biologically active material is selected from the group consisting of paclitaxel, paclitaxel analogues, paclitaxel derivatives, and combinations thereof; and
wherein at least a portion of the inner surface and side surface is covered by a second coating comprising a second polymeric material and the inner surface and side surface are substantially free of the biologically active material, and
wherein the first coating and the second coating form a continuous coating disposed on the struts.

12. A method of coating a medical device comprising:
(a) providing a prefabricated medical device having a tubular sidewall, wherein the sidewall comprises a plurality of struts each having an outer surface, an inner surface opposite the outer surface, and at least one side surface adjacent to the inner surface and the outer surface and connecting the inner surface and outer surface;
(b) applying to the outer surface or the inner surface a first coating comprising a first coating material dispersed in a first polymeric material; and
(c) applying to either the outer surface or the inner surface, that is not covered by the first coating, and side surfaces a second coating comprising a second polymeric material, wherein the second coating is substantially free of the first coating material material, in a manner such that the first coating and second coating form a continuous coating disposed on the struts and such that the surface covered with the first coating is substantially free of the second coating and that the surface covered by the second coating is substantially free of the first coating.

13. The method of 12, wherein the medical device is a stent.

14. The method of 12, further comprising applying the first coating on the outer surface by rolling the medical device on a substrate coated with the first coating.

15. The method of 12, further comprising applying the second coating to the inner surface and the side surface by spray-coating.

16. The method of 12, further comprising masking either the inner or outer surface while a coating is applied to the opposing surface.

17. The method of claim 12, wherein the first coating material is a biologically active material.

18. The method of 17, wherein the first coating material is a biologically active material is selected from the group consisting of anti-thrombogenic agents, anti-angiogenesis agents, anti-proliferative agents, growth factors, and radiochemicals.

19. The method of claim 18, wherein the anti-proliferative agent is selected from the group consisting of paclitaxel, paclitaxel analogues, paclitaxel derivatives, and combinations thereof.

20. The method of 12, wherein the first and second polymeric materials are selected from the group consisting of styrene-isobutylene-styrene, polyurethanes, silicones, polyesters, polyolefins, polyisobutylene, ethylene-alphaolefin copolymers, acrylic polymers and copolymers, vinyl halide polymers, polyvinyl ethers, polyvinylidene halides, polyacrylonitrile, polyvinyl ketones, polyvinyl aromatics, polyvinyl esters, copolymers of vinyl monomers, copolymers of vinyl monomers and olefins, polyamides, alkyd resins, polycarbonates, polyoxymethylenes, polyimides, polyethers, epoxy resins, polyurethanes, rayon-triacetate, cellulose, cellulose acetate, cellulose butyrate, cellulose acetate butyrate, cellophane, cellulose nitrate, cellulose propionate, cellulose ethers, carboxymethyl cellulose, collagens, chitins, polylactic acid, polyglycolic acid, polylactic acid-polyethylene oxide copolymers, ethylene-propylene-diene rubbers, fluorosilicones, polyethylene glycol, polysaccharides, phospholipids, and combinations thereof.

21. The method of 12, wherein the first and second polymeric materials are biocompatible.

22. The method of 12, wherein the second polymeric material is the same as the first polymeric material.

23. The method of 12, wherein the inner surface is covered by the second coating and the second polymeric material is harder than the first polymeric material.

24. A method of coating an expandable stent having a sidewall comprising a plurality of struts, each having an outer surface, an inner surface opposite the outer surface, and at least one side surface adjacent to the outer surface and the inner surface and connecting the outer surface and the inner surface, the method comprising:
(a) applying to the outer surface a first coating comprising a biologically active material dispersed in a first polymeric material, wherein the biologically active material is selected from the group consisting of paclitaxel, paclitaxel analogues, paclitaxel derivatives, and combinations thereof; and
(b) applying to the inner surface and the side surface a second coating comprising a second polymeric material in a manner such that the first coating and the second coating form a continuous coating disposed on the struts and the inner surface and side surface are substantially free of the biologically active material.
